(19) United States
(12) Patent Application Publication
(10) Pub. No.: US 2009/0233303 A1
(43) Pub. Date: Sep. 17, 2009

(54) METHODS FOR ASSESSING BREAKDOWN PRODUCTS AND STABILITY OF SIRNA AND OTHER TARGET OLIGONUCLEOTIDES

(75) Inventor: John F. Thompson, Warwick, RI (US)

Correspondence Address: COOLEY GODWARD KRONISH LLP;ATTN: Patent Group, Suite 1100, 777 - 6th Street, NW, WASHINGTON, DC, 20001, US

(73) Assignee: HELICOS BIOSCIENCES CORPORATION, CAMBRIDGE, MA (US)

(21) Appl. No.: 12/403,597

(22) Filed: Mar. 13, 2009

Related U.S. Application Data
(60) Provisional application No. 61/036,326, filed on Mar. 13, 2008.

(51) Int. Cl. C12Q 1/68

(52) U.S. Cl. 435/6

(57) ABSTRACT

The disclosure provides methods and compositions for assessing by sequencing the type and quantity of in vivo or in vitro breakdown products and stability of nucleic acids such as small interfering RNA (siRNA) and other target oligonucleotides. In general, the disclosed methods involve the use of tester oligonucleotides that comprise a double-stranded region, an optional loop (in the case of hairpin testers), and a single-stranded 3′ overhang that is complementary to the full-length and a shortened target oligonucleotide. A tester is designed so that the 3′ end of a respective target oligonucleotide anneals to the overhang immediately adjacent to the 5′ end of the tester. The juxtaposed ends of the tester and target at adjacent positions allow for a ligase to ligate the chain if there is a match between a tester and its respective target. By sequencing the ligated product in the region of the ligation site, one may determine the sequence of the 3′ end of the target oligonucleotide or of the entire target and its relative amount in the sample.

This application claims priority to US application No. 61/036,326 filed on Mar. 13, 2008.

### TECHNICAL FIELD

The invention is in the field of molecular biology and relates to methods for nucleic acid analysis. In particular, the invention relates to methods of assessing the type and quantity of breakdown products and the stability of nucleic acids such as small interfering RNA (siRNA) and other oligonucleotides.

### BACKGROUND OF THE INVENTION

siRNA, anti-sense, and other oligonucleotides are becoming used increasingly frequently as therapeutics for alleviating disease. Understanding the stability and breakdown products of these oligonucleotides both during storage or after administration to animals, or humans, is a challenge due to their large size and complexity. Accordingly, there is a need for methods of assessing the type and quantity of breakdown products and the stability of nucleic acids such as small interfering RNA (siRNA) and other oligonucleotides, particularly, methods for assessing in vivo stability of such oligonucleotides using biological samples from the treated subjects.

### SUMMARY OF THE INVENTION

The invention provides methods and compositions for high-throughput assessment of a biological sample by sequencing the type and quantity of breakdown products and stability of nucleic acids such as small interfering RNA (siRNA) and other oligonucleotides. The methods may be used, for example, to obtain information about the amount and relative contribution and the identity of degradation products formed upon in vivo administration, e.g., following a time course; for comparisons across subjects; or for testing pharmacokinetics of various forms of target oligonucleotides in drug design, as well as for drug safety or efficacy analyses. The oligonucleotides that are being assessed by methods of the invention are generally referred herein as “target oligonucleotides” (or “targets”). Unless otherwise stated, this term refers to both the full-length original target oligonucleotides as well as to their breakdown products, such as shortened target oligonucleotides, including 3′- and 5′- truncated oligonucleotides.

In general, methods of the invention involve the use of “tester oligonucleotides” (or “testers”) that comprise a double-stranded region, an optional loop (in the case of hairpin testers), and a single-stranded 3′ overhang that is complementary to either a full-length or a shortened target oligonucleotide. A tester is designed so that the 3′ end of a respective target nucleotide anneals to the overhang immediately adjacent to the 5′ end of the tester. The juxtaposed ends of the tester and target being at adjacent positions allow for a ligase to ligate the chain if there is a match between a tester and its respective target. No match or a match producing a gap will not ligate, resulting in a “blank” tester. Thereafter, by sequencing the ligated product in the region of the ligation site, one may determine the sequence of the 3′ end of the target oligonucleotide or of the entire target (whether full-length or shortened), its relative amount in the sample, and if desired, the identity of the tester. In preferred embodiments, a biological sample comprising a target oligonucleotide is contacted with multiple testers recognizing varying truncated forms and/or the full-length target nucleotide. By determining which testers successfully ligate to their respective targets, one may determine the spectrum of degradation products found in the sample.

Accordingly, the methods of invention include:
a) contacting a sample comprising a target oligonucleotide with one or more tester oligonucleotides under annealing conditions;
b) ligating the 5′ end of the tester oligonucleotide to the 3′ end of the target oligonucleotide if one is annealed adjacent to the 5′ end of the tester oligonucleotide; and
c) sequencing at least a portion of the tester oligonucleotide proximal to the 5′ end, and if present, at least a portion of the 3′ end of the target nucleotide ligated to the tester oligonucleotide, thereby to detect the target oligonucleotide or its breakdown product in the sample.

The samples being evaluated may be obtained from a subject which has been administered the full-length target oligonucleotide and may include samples obtained from the blood, urine, or other bodily fluid or tissue of the subject.

The invention further provides compositions for use in the methods of the invention, including individual testers and kits that include multiple testers (e.g., 10 or more testers to various degradation products of a target oligonucleotide), optionally, in combination with the target oligonucleotide. The testers of the invention may include a) a nucleotide sequence barcode, b) a cleavage (e.g., restriction) site, and c) a universal capture sequence, a universal primer, a complement of the universal capture sequence, and/or a complement of the universal primer.

In some embodiments, the sequencing is performed by synthesis. In preferred embodiments, the sequencing is performed at a single molecule resolution. The target oligonucleotides may be analyzed by sequencing without being pre-amplified prior to sequencing. The analysis may include determining the degradation site(s) in the target, comparing relative amounts of degradation products, determining a pharmacokinetic profile of the target oligonucleotide, etc.

### DETAILED DESCRIPTION OF THE INVENTION

The invention provides methods and compositions for analyzing stability and/or breakdown of a target oligonucleotide. In general, the method of the invention includes:
a) contacting a sample comprising a target oligonucleotide (e.g., an siRNA) with one or more tester oligonucleotides under annealing conditions;
b) ligating the 5′ end of the tester oligonucleotide to the 3′ end of the target oligonucleotide if one is annealed to the tester oligonucleotide adjacent to the tester's 5′ end; and

c) sequencing at least a portion of the tester oligonucleotide proximal to the 5′ end, and if present, at least a portion of the 3′ end of the target oligonucleotide ligated to the tester oligonucleotide, thereby to detect the target oligonucleotide or its breakdown product in the sample.

In more specific embodiments, the method of the invention includes:
a) administering a target oligonucleotide to a subject;
b) obtaining a sample from the subject;
c) contacting the sample with a plurality of tester oligonucleotides under annealing conditions;
d) ligating the 5′ end of the tester oligonucleotide to the 3′ end of the full-length or the 3′-truncated target oligonucleotide annealed to the tester oligonucleotide;
e) immobilizing the ligated tester onto a support; and
f) conducting a sequencing by synthesis reaction so as to sequence at least a portion of the tester oligonucleotide proximal to the 5′ end, and if present, at least a portion of the ligated target oligonucleotide, thereby detecting the full-length and/or the 3′-truncated target oligonucleotide in the sample.

In steps c) and d), a plurality of tester oligonucleotides are placed in contact with a sample that comprises the full-length and/or shortened target oligonucleotides, raised to a temperature that would allow denaturation of any target oligonucleotide's secondary structure, and then annealed to the tester oligonucleotides. After annealing, the mixture may be exposed to T4 DNA ligase or a similar enzyme that allows ligation of DNA-DNA or RNA-DNA when situated in exact complementarity in a double stranded structure. Inexact matches will not ligate. If desired, the denaturation/annealing/ligation conditions can be repeated for multiple rounds in order to maximize the collection of perfect matches. After a suitable number of rounds of ligation, the tester oligonucleotides can be captured by hybridization or by some other means of affinity capture designed into their structure. The hairpin can be cleaved by the restriction endonuclease site designed into the original tester molecules to allow easier capture or sequencing. Such molecules can then be captured onto a surface for direct sequencing or copied first into DNA and then sequenced. Both the 5′ and 3′ ends of the target sequence can then be determined via direct sequencing with the ratios of each determined via counting. Since hybridization and other parameters may vary depending on the exact sequence of the target molecules, controls could be run to assess whether normalization of results is necessary. Thus, some embodiments further include determining the efficiency of the detection for the detected target oligonucleotide(s), e.g., by spiking the starting sample with a known amount of detected target oligonucleotides.

Additional methods and compositions of the invention are described in detail below.

### Tester Oligonucleotides

In accordance with the present invention, tester oligonucleotides comprise a double-stranded region, an optional loop (in the case of a hairpin structure), and a single-stranded 3′ overhang region that is complementary to a full-length and a shortened target oligonucleotide. In the case of non-hairpin structures, both ends of the double-stranded region may have overhangs. In some embodiments, a tester set includes one tester oligonucleotide for each target oligonucleotide shortened by successive nucleotides at the 3′ end. In some embodiments, the 5′ end of each tester oligonucleotide is designed to be different than the next 3′ position of the target oligonucleotide to prevent alternative base pairing between different length target sequences.

The length of the double-stranded region of the testers may vary. In general, it should be of sufficient length to provide a relatively stable structure. For example, the double-stranded region may be 10-100, 10-75, 10-50, 15-50, 15-35, 15-25, or about 20 bps long. In preferred embodiments, the double-stranded region has a GC content of above 40%, above 45%, above 50%, or above 60%. Accordingly, tester oligonucleotides with higher melting temperatures may be preferred. In some embodiments, a tester oligonucleotide has a melting temperature higher than 65, 67, 70, 72, 75, 77, or 80° C.

The length of the single-stranded 3′ overhang may also vary and be tailored in accordance with the length of the respective target oligonucleotide. For example, the length of the single-stranded region may be 1-50, 1-40, 1-35, 1-25, 1-20, or about 20 nts long.

In some embodiments, tester oligonucleotides may have a hairpin structure such as illustrated in Example 1. Example 1 also provides specific illustrative embodiments. The length of the loop region may also vary and may be, for example, 1, 2, 3, 5, 5-30, or 5-20 nts.

Barcodes—In some embodiments, tester oligonucleotides may contain one or more barcode sequences. The barcode may identify the sample, e.g., by its serial number, source (multiple individuals, samples from multiple timepoints), and/or location during processing (e.g., a plate-specific barcode, a batch-specific barcode), different treatment conditions, disease, tissue, etc. For example, the barcode may identify a compound tested in a given sample from a library of compounds. As another example, the barcode may correspond to the source of tissue or cells from a tissue/cell bank. In general, the term “barcode” refers to known nucleic acid sequences that are specifically added to naturally occurring sequences to serve as unique identifiers of the sequence identity, origin, or source. Examples of barcodes are described, for example, in Shoemaker et al. (1996) Nature Genetics, 14:450; Parameswaran et al. (2007) Nucleic Acids Res., 35:e130; and in the Example. Barcodes are typically less than 20-nucleotides long and are designed to be maximally different yet still retain similar hybridization properties. In some embodiments, a barcode used in the methods of the invention may be, for example, 4-25, 6-18, 8-14, or 10-12 nts long. Desirable barcode sequences have no homopolymers (2 or more of the same base in a row), have sequence edit distances greater than 2 or more bases apart in the encoded barcode (so that the barcodes are error tolerant, i.e., sequencing-by-synthesis process reading errors do not convert a barcode from one to another), and have sequences which are normalized for growth rate in the sequencing-by-synthesis process.

Cleavage sites—In some embodiments, the tester oligonucleotides may contain one or more cleavage sites such as restriction sites positioned, preferably, in the double-stranded region. Examples of restriction enzymes and their respective recognition sites that can be used in the present invention include those found in, e.g., Restriction Endonucleases (Nucleic Acids and Molecular Biology) by Pingoud (Editor), Springer; 1 ed. (2004)). Many restriction enzymes are available commercially, e.g., from New England BioLabs (Beverly, Mass.).

Universal Primers and Capture Sequences—In some embodiments, the tester oligonucleotides may contain a universal capture sequence, a universal primer, a complement of the universal capture sequence, and/or a complement of the universal primer. These elements are preferably positioned in the double-stranded region. In some embodiments, a universal primer complement is used for reverse transcription of the ligated product, or a substrate for a DNA polymerase (e.g., Klenow exo⁻) for direct sequencing by synthesis. In certain embodiments, the universal primer is positioned in the double-stranded region between the barcode and the restriction site, as illustrated in Example 1 (denoted as NNNNNN).

In some embodiments, the primer may function as a universal capture sequence which is complementary to a sequence attached to a support. In some embodiments, the target/tester ligated product, or its copy, is polyadenylated as described in the Examples. Thus, in some embodiments, the capture sequence is polyNₙ, wherein N is U, A, T, G, or C, n≧5, e.g., 10-30, 15-25, e.g., about 20. For example, the capture sequence could be polyA₂₀₋₃₀ or its complement.

### Target Oligonucleotides and Samples

Target oligonucleotides can come from a variety of sources. For example, nucleic acids can be naturally occurring DNA or RNA (e.g., mRNA or non-coding RNA) isolated from any source, recombinant molecules, cDNA, or synthetic analogs. For example, the target oligonucleotide may include whole genes, gene fragments, exons, introns, regulatory elements (such as promoters, enhancers, initiation and termination regions, expression regulatory factors, expression controls, and other control regions), DNA comprising one or more single-nucleotide polymorphisms (SNPs), allelic variants, and other mutations. The target oligonucleotide can be tRNA, rRNA, a ribozyme, but preferably, is antisense RNA, microRNA, or siRNA. siRNA is described for example, in U.S. Pat. Nos. 6,506,559, 7,056,704, 7,078,196, 6,107,094, 6,573,099, and European Patent No. 1,144,623. The length of the target nucleic acid may vary. siRNA are small double-stranded RNAs generally about 15-25 nucleotides long, most commonly, 15-23, 19-23, or about 21-23 nucleotides long. However, the target oligonucleotides may also be longer, for example, at least 50, 100, 300, 350, 400, 450, 500, 1000 nucleotides or longer. The methods of the invention can be applied to a target oligonucleotide of any length that could have its 3′ end annealed to a given tester. (The methods generally do not require that the entire length of the target be sequenced.)

In some embodiments, methods of the invention include assaying an in vitro sample comprising a target oligonucleotide, for example, for quality control in the manufacturing and/or during storage.

In some embodiments, methods of the invention include administering a full-length target oligonucleotide to a subject and then obtaining a sample from the subject. Target oligonucleotides may be obtained from samples obtained from whole organisms, organs, tissues, cells, or biological fluids (urine, blood, lymph, etc.) from different stages of development, differentiation, or disease state, and from different species (e.g., human and non-human animals, primates, rodents, plants). Various methods for extraction of nucleic acids from biological samples are known (see, e.g., Nucleic Acids Isolation Methods, Bowein (ed.), American Scientific Publishers, 2002). The sample pre-purified prior to the addition of tester oligonucleotides, for example, by ethanol precipitation of nucleic acid or other suitable methods.

In some embodiments, the methods of the invention are used to compare the amount of the target oligonucleotide relative to the same in another sample. The second sample may be obtained from the same subject after a period of time from obtaining the first sample. The second sample may also be obtained from another subject that was administered the same target oligonucleotide or a modified version of the target oligonucleotide (e.g., containing a methylation site or a nucleotide substitution) or a second substantially different candidate oligonucleotide.

The analysis of samples may include determining the degradation site(s) in the target, comparing relative amounts of degradation products, determining a pharmacokinetic profile of the target oligonucleotide, etc. For example, in some embodiments, one determines the amounts of the full-length target oligonucleotide and the amount of at least one shortened target oligonucleotide in the sample.

The invention further provides compositions for use in the methods of the invention, including individual testers, and kits that include multiple testers (e.g., 2, 3, 4, 5, 6, 7, 8, 9, 10, 11, 12, 13, 14, 15, 16, 17, 18, 19, 20 or more testers directed to various degradation products of a target oligonucleotide), optionally, in combination with the target oligonucleotide. In some preferred embodiments, tester nucleotides have a hairpin structure and contain a barcode, a universal primer, and a restriction site in the order as illustrated in Example 1 (e.g., from the overhang towards the loop).

### Sequencing Platforms

A number of initiatives are currently underway to obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

The invention can be used on any suitable sequencing-by-synthesis platform. As described above, four major sequencing-by-synthesis platforms are currently available: the Genome Sequencers from Roche/454 Life Sciences, the 1G Analyzer from Illumina/Solexa, the SOLiD system from Applied BioSystems, and the Heliscope system from Helicos Biosciences. Sequencing-by-synthesis platforms have also been described by Pacific BioSciences and VisiGen Biotechnologies. Each of these platforms can be used in the methods of the invention. In some embodiments, the sequencing platforms used in the methods of the present invention have one or more of the following features:
1) four differently optically labeled nucleotides are utilized (e.g., 1G Analyzer, Pacific BioSciences, and Visigen);
2) sequencing-by-ligation is utilized (e.g., SOLiD);
3) pyrophosphate detection is utilized (e.g., Roche/454);
4) four similarly optically labeled nucleotides are utilized (e.g., Helicos); and
5) fluorescent energy transfer (FRET) is utilized (e.g., Visigen).

In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer. In some embodiments, the capture sequence is polyNₙ, wherein N is U, A, T, G, or C, n≧5, e.g., 20-70, 40-60, e.g., about 50. For example, the capture sequence could be polyT₄₀₋₅₀ or its complement.

As an alternative to a capture sequence, a member of a coupling pair (such as, e.g., antibody/antigen, receptor/ligand, or the avidin-biotin pair as described in, e.g., US Patent Application No. 2006/0252077) may be linked to each fragment to be captured on a surface coated with a respective second member of that coupling pair.

The support may be, for example, a glass surface such as described in, e.g., US Patent App. Pub. No. 2007/0070349. The surface may be coated with an epoxide, polyelectrolyte multilayer, or other coating suitable to bind nucleic acids. In preferred embodiments, the surface is coated with epoxide and a complement of the capture sequence is attached via an amine linkage. The surface may be derivatized with avidin or streptavidin, which can be used to attach to a biotin-bearing target nucleic acid. Alternatively, other coupling pairs, such as antigen/antibody or receptor/ligand pairs, may be used. The surface may be passivated in order to reduce background. Passivation of the epoxide surface can be accomplished by exposing the surface to a molecule that attaches to the open epoxide ring, e.g., amines, phosphates, and detergents.

Subsequent to the capture, the sequence may be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide may be incorporated and multiple lasers may be utilized for stimulation of incorporated nucleotides.

The following Examples provide illustrative embodiments of the invention and do not limit the invention in any way.

### EXAMPLES

### Example 1

### Illustrative Target and Testers

As an illustration of the methods of the invention, an siRNA sequence from U.S. Pat. No. 7,176,304 (sequences 2585 and 2588) and one possible set of degradation products are shown below with representative tester sequences for the top strand. A second set of constructs could be made for the bottom strand. If the target oligonucleotide is a hairpin with a longer overhang, one set of testers could be used for both strands.

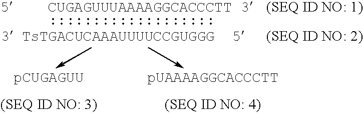

The following hairpin Tester 1 and a set of up to 20 with varying overhang lengths could be used to monitor the full spectrum of potential cleavages.

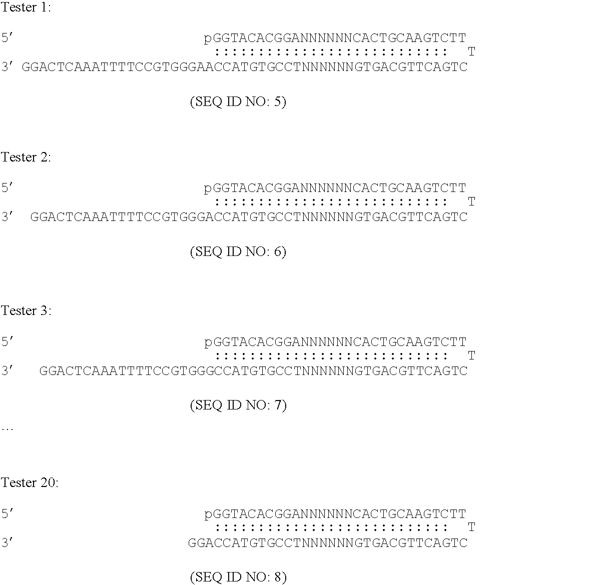

In the above testers, the barcode is set to CACGGA, and restriction enzyme site is Btsl (CACTGC) to allow cutting within the non-desired primer strand but other sequences could be used. Alternatively, the N primer and restriction site could be removed and a long polyA stretch could be inserted in the hairpin loop for surface capture. Hybridization products for the full-length oligonucleotides and two degradation products annealed to Tester 1 are illustrated below.

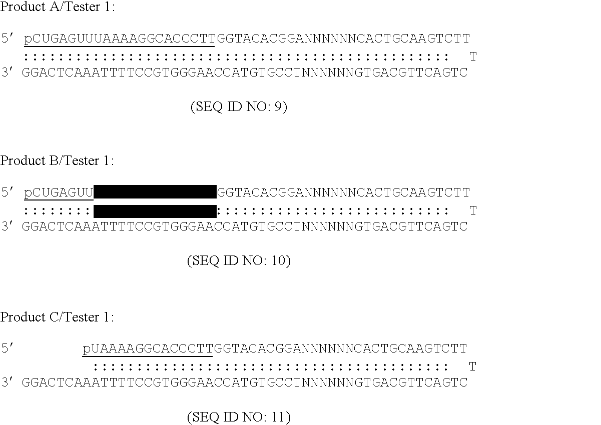

In this example, only the full-length parent (Product A) and degradation product C could be ligated successfully to tester 1. Product B can be ligated to a different tester. None of the other target sequences will ligate normally to testers that have longer overhangs than the target sequences themselves.

Upon cutting with a restriction endonuclease (preferably with staggered cut within capture sequence as with Btsl), the strands are captured onto a surface. Sequencing is performed using reverse transcriptase, followed by binning by both 5′ and 3′ ends.

Tester 1 will yield three different sequences:

(SEQ ID NO: 12)

pCUGAGUUUAAAAGGCACCCTTGGTACACGGANNNNNN

(SEQ ID NO: 13)

pGGTACACGGANNNNNN

(SEQ ID NO: 14)

pUAAAAGGCACCCTTGGTACACGGANNNNNN

Tester 2 will yield only the original sequence:

| | |
|---|---|
| pGGGTACACGGANNNNNN | (SEQ ID NO: 15) |

Tester 15 will yield two different sequences:

(SEQ ID NO: 16)

pCUGAGUUGGCTTACAGGCTTGGTACACGGANNNNNN

(SEQ ID NO: 17)

pGGCTTACAGGCTTGGTACACGGANNNNNN

The primer is then hybridized to the NNNNNN sequence and extended with reverse transcriptase. PolyA tail is added to the 3′ end of newly transcribed DNA. Tailed DNA are captured onto a surface and sequenced up from the polyT capture sequence (or another suitable capture sequence).

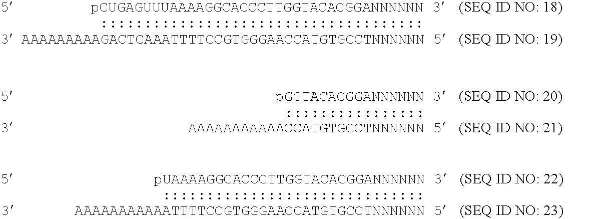

### Example 2

### Single Molecule Sequencing

Epoxide-coated glass slides are prepared for oligo attachment. Epoxide-functionalized 40 mm diameter #1.5 glass cover slips (slides) are obtained from Erie Scientific (Salem, N.H.). The slides are preconditioned by soaking in 3×SSC for 15 minutes at 37° C. Next, a 500-pM aliquot of 5′ aminated capture oligonucleotide is incubated with each slide for 30 minutes at room temperature in a volume of 80 ml. The slides are then treated with phosphate (1 M) for 4 hours at room temperature in order to passivate the surface. The slides are then stored in 20 mM Tris, 100 mM NaCl, 0.001% Triton® X-100, pH 8.0 at 4° C. until they are used for sequencing.

For the illustration of the sequencing process, see, e.g., U.S. patent application Ser. No. 12/043,033 (FIGS. 1A and 1B). For sequencing, the slide is placed in a modified FCS2 flow cell (Bioptechs, Butler, Pa.) using a 50-μm thick gasket. The flow cell is placed on a movable stage that is part of a high-efficiency fluorescence imaging system built based on a Nikon TE-2000 inverted microscope equipped with a total internal reflection (TIR) objective. The slide is then rinsed with HEPES buffer with 100 mM NaCl and equilibrated to a temperature of 50° C. The oligonucleotides to be sequenced are labeled with Cy3 at the 5′ end, and then diluted in 3×SSC to a final concentration of 200 pM (each). A 100-μl aliquot is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains the oligonucleotides/primer template duplex randomly bound to the glass surface. The temperature of the flow cell is then reduced to 37° C. for sequencing and the objective is brought into contact with the flow cell.

Further, cytosine triphosphate, guanidine triphosphate, adenine triphosphate, and uracil triphosphate, each having a cleavable cyanine-5 label (at the 7-deaza position for ATP and GTP and at the C5 position for CTP and UTP (PerkinElmer) are stored separately in the buffer containing 20 mM Tris-HCl, pH 8.8, 50 μM MnSO₄, 10 mM (NH₄)₂SO₄, 10 mM HCl, and 0.1% Triton X-100, and 50 U Klenow exo⁻ polymerase (NEB).

Sequencing proceeds as follows. First, initial imaging is used to determine the positions of duplex on the epoxide surface. The Cy3 label attached to the synthetic oligo fragments is imaged by excitation using a laser tuned to 532 nm radiation (Verdi V-2 Laser, Coherent, Santa Clara, Calif.) in order to establish duplex position. For each slide only single fluorescent molecules that are imaged in this step are counted. Imaging of incorporated nucleotides as described below is accomplished by excitation of a cyanine-5 dye using a 635-nm radiation laser (Coherent). 100 nM Cy5-CTP is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 μl volumes each, followed by 150 mM HEPES/150 mM NaCl/pH 7.0 (“HEPES/NaCl”) (10 times at 60 μl volumes). An oxygen scavenger containing 30% acetonitrile and scavenger buffer (134 μl 150 mM HEPES/100 mMNaCl, 24 μl 100 mM Trolox in 150 mM MES, pH 6.1, 10 μl 100 mM DABCO in 150 mM MES, pH 6.1), 8 μl 2 M glucose, 20 μl 50 mM Nal, and 4 μl glucose oxidase (USB) is next added. The slide is then imaged (100 frames) for 2 seconds using an Inova 301K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 μl) and HEPES/NaCl (60 μl). Next, the cyanine-5 label is cleaved off the incorporated CTP by introduction into the flow cell of 50 mM TCEP/250 mM Tris, pH 7.6/100 mM NaCl for 5 minutes, after which the flow cell is rinsed 5 times each with SSC/HEPES/SDS (60 μl) and HEPES/NaCl (60 μl). The remaining nucleotide is capped with 50 mM iodoacetamide/100 mM Tris, pH 9.0/100 mM NaCl for 5 minutes followed by rinsing 5 times each with SSC/HEPES/SDS (60 82 l) and HEPES/NaCl (60 μl). The scavenger is applied again in the manner described above, and the slide is again imaged to determine the effectiveness of the cleave/cap steps and to identify non-incorporated fluorescent objects.

The procedure described above is then conducted 100 nM Cy5-dATP, followed by 100 nM Cy5-dGTP, and finally 100 nM Cy5-dUTP. Uridine may be used instead of Thymidine due to the fact that the Cy5 label is incorporated at the position normally occupied by the methyl group in Thymidine triphosphate, thus turning the dTTP into dUTP. The procedure (expose to nucleotide, polymerase, rinse, scavenger, image, rinse, cleave, rinse, cap, rinse, scavenger, final image) is repeated for a total of 48 cycles.

Once the desired number of cycles is completed, the image stack data (i.e., the single-molecule sequences obtained from the various surface-bound duplexes) are aligned to the reference barcode sequences. The individual single molecule sequence read lengths obtained range from 2 to 16 consecutive nucleotides with about 12.6 consecutive nucleotides being the average length and only those greater than 9 bases in length with fewer than 2 errors where used in the final analysis.

Once the desired number of cycles is completed, the image stack data (i.e., the single-molecule sequences obtained from the various surface-bound duplex) are aligned to the reference sequence. Only the individual single molecule sequence read lengths obtained ranging from 6 and above are analyzed. A missing base error is detected, when the single molecule sequence contains a gap (of one or more nucleotides) compared to the reference sequence.

All publications, patents, patent applications, and biological sequences cited in this disclosure are incorporated by reference in their entirety.

1. A method for analyzing stability and/or breakdown of a target oligonucleotide, the method comprising:
a) contacting a sample comprising a full-length or shortened target oligonucleotide with one or more tester oligonucleotides under annealing conditions, said tester nucleotides each comprising a double-stranded region, an optional loop, and a single-stranded 3′ overhang complementary to the full-length or a shortened target oligonucleotide;
b) ligating the 5′ end of the tester oligonucleotide to the 3′ end of the oligonucleotide if one is annealed adjacent to the 5′ end of the tester oligonucleotide; and
c) sequencing at least a portion of the tester oligonucleotide proximal to the 5′ end, and if present, at least a portion of the 3′ end of the target oligonucleotide ligated to the tester oligonucleotide, thereby to detect the target oligonucleotide or its breakdown product in the sample.

2. The method of claim 1, wherein the method further comprises:
i) administering the full-length target oligonucleotide to a subject; and
ii) obtaining the sample from the subject.

3. The method of claim 1, wherein the method further comprises:
iii) pre-purifying the sample prior to step a).

4. The method of claim 1, wherein the full-length target oligonucleotide is 100 or fewer nucleotides long.

5. The method of claim 1, wherein the target oligonucleotide is siRNA.

6. The method of claim 1, wherein the sample is contacted in step c) with a plurality of tester oligonucleotides with varying overhangs complementary to the target oligonucleotides shortened at the 3′ end by one or more nucleotides.

7. The method of claim 1, wherein the sequencing is sequencing by synthesis.

8. The method of claim 1, wherein the target oligonucleotides are not amplified prior to sequencing.

9. The method of claim 7, wherein the sequencing by synthesis is performed using a DNA polymerase or a reverse transcriptase.

10. The method of claim 7, wherein the sequencing by synthesis is performed at a single molecule resolution.

11. The method of claim 7, wherein one or more of the ligated target oligonucleotides are copied by reverse transcriptase for the sequencing in step c).

12. The method of claim 1, wherein one or more tester oligonucleotides have a hairpin structure.

13. The method of claim 1, wherein one or more tester oligonucleotides comprise one or more of the following: a nucleotide sequence barcode, a restriction site, a universal capture sequence, a universal primer, a complement of the universal capture sequence, and/or a complement of the universal primer.

14. The method of claim 1, wherein the method comprises multiple cycles of denaturation/annealing/ligation prior to step c).

15. The method of claim 1, further comprising determining the efficiency of detection for the detected target oligonucleotide(s).

16. The method of claim 1, comprising determining the amount of the target oligonucleotide relative to the same in a second sample.

17. The method of claim 16, wherein the second sample is obtained from the same subject after a period of time from obtaining the first sample.

18. The method of claim 16, wherein the second sample is obtained from another subject that was administered a modified target oligonucleotide.

19. The method of claim 1, further comprising d) determining the amount of the full-length target oligonucleotide and the amount of at least one shortened target oligonucleotide in the sample.

20. An apparatus adapted for performing the method of claim 1.

21. An apparatus adapted for performing the method of claim 7.

22. An apparatus adapted for performing the method of claim 10.

23. A method for analyzing stability and/or breakdown of siRNA, the method comprising:
a) administering siRNA to a subject;
b) obtaining a sample from the subject;
c) contacting the sample with a plurality of tester oligonucleotides under annealing conditions, said tester nucleotides each comprising a double-stranded region, an optional loop region, and a single-stranded 3′ overhang complementary to the full-length siRNA or a 3′-truncated siRNA;
d) ligating the 5′ end of the tester oligonucleotide to the 3′ end of the full-length or the 3′-truncated siRNA annealed to the tester oligonucleotide;
e) immobilizing the ligated tester onto a support; and
f) conducting a sequencing-by-synthesis reaction so as to sequence at least a portion of the tester oligonucleotide proximal to the 5′ end, and if present, at least a portion of the ligated siRNA, thereby detecting the full-length and/or the 3′-truncated siRNA in the sample.

24. A kit comprising a plurality of hairpin tester oligonucleotides, each tester oligonucleotide comprising a single-stranded 3′ overhang complementary to a target siRNA or 3′-truncated products of the target siRNA and, optionally, further comprising the target siRNA.

25. The kit of claim 24, wherein the kit comprises about 15 tester oligonucleotides with varying 3′ overhangs complementary to the 3′-truncated products of varying length.


SEQUENCE LISTING (23 sequences)

SEQ ID NO: 1 - DNA, 21 bp, Artificial Sequence
  Description of Combined DNA/RNA Molecule Synthetic oligonucleotide
cugaguuuaaaaggcaccctt

SEQ ID NO: 2 - DNA, 21 bp, Artificial Sequence
  Description of Combined DNA/RNA Molecule Synthetic oligonucleotide
gggugccuuuuaaacucagtt

SEQ ID NO: 3 - RNA, 7 nt, Artificial Sequence
  Description of Artificial Sequence Synthetic oligonucleotide
cugaguu

SEQ ID NO: 4 - DNA, 14 bp, Artificial Sequence
  Description of Combined DNA/RNA Molecule Synthetic oligonucleotide
uaaaaggcaccctt

SEQ ID NO: 5 - DNA, 81 bp, Artificial Sequence
  Description of Artificial Sequence Synthetic oligonucleotide
ggtacacggannnnnncactgcaagtctttctgacttgcagtgnnnnnntccgtgtaccaagggtgccttttaaactcagg

SEQ ID NO: 6 - DNA, 80 bp, Artificial Sequence
  Description of Artificial Sequence Synthetic oligonucleotide
ggtacacggannnnnncactgcaagtctttctgacttgcagtgnnnnnntccgtgtaccagggtgccttttaaactcagg

SEQ ID NO: 7 - DNA, 79 bp, Artificial Sequence
  Description of Artificial Sequence Synthetic oligonucleotide
ggtacacggannnnnncactgcaagtctttctgacttgcagtgnnnnnntccgtgtaccgggtgccttttaaactcagg

SEQ ID NO: 8 - DNA, 62 bp, Artificial Sequence
  Description of Artificial Sequence Synthetic oligonucleotide
ggtacacggannnnnncactgcaagtctttctgacttgcagtgnnnnnntccgtgtaccagg

SEQ ID NO: 9 - DNA, 102 bp, Artificial Sequence
  Description of Combined DNA/RNA Molecule Synthetic oligonucleotide
cugaguuuaaaaggcacccttggtacacggannnnnncactgcaagtctttctgacttgcagtgnnnnnntccgtgtaccaagggtgccttttaaactcagg

SEQ ID NO: 10 - DNA, 81 bp, Artificial Sequence; identical to SEQ ID NO: 5
  Description of Artificial Sequence Synthetic oligonucleotide

SEQ ID NO: 11 - DNA, 95 bp, Artificial Sequence
  Description of Combined DNA/RNA Molecule Synthetic oligonucleotide
uaaaaggcacccttggtacacggannnnnncactgcaagtctttctgacttgcagtgnnnnnntccgtgtaccaagggtgccttttaaactcagg

SEQ ID NO: 12 - DNA, 37 bp, Artificial Sequence
  Description of Combined DNA/RNA Molecule Synthetic oligonucleotide
cugaguuuaaaaggcacccttggtacacggannnnnn

SEQ ID NO: 13 - DNA, 16 bp, Artificial Sequence
  Description of Artificial Sequence Synthetic oligonucleotide
ggtacacggannnnnn

SEQ ID NO: 14 - DNA, 30 bp, Artificial Sequence
  Description of Combined DNA/RNA Molecule Synthetic oligonucleotide
uaaaaggcacccttggtacacggannnnnn

SEQ ID NO: 15 - DNA, 17 bp, Artificial Sequence
  Description of Artificial Sequence Synthetic oligonucleotide
gggtacacggannnnnn

SEQ ID NO: 16 - DNA, 36 bp, Artificial Sequence
  Description of Combined DNA/RNA Molecule Synthetic oligonucleotide
cugaguuggcttacaggcttggtacacggannnnnn

SEQ ID NO: 17 - DNA, 29 bp, Artificial Sequence
  Description of Artificial Sequence Synthetic oligonucleotide
ggcttacaggcttggtacacggannnnnn

SEQ ID NO: 18 - DNA, 37 bp, Artificial Sequence; identical to SEQ ID NO: 12
  Description of Combined DNA/RNA Molecule Synthetic oligonucleotide

SEQ ID NO: 19 - DNA, 46 bp, Artificial Sequence
  Description of Artificial Sequence Synthetic oligonucleotide
nnnnnntccgtgtaccaagggtgccttttaaactcagaaaaaaaaa

SEQ ID NO: 20 - DNA, 16 bp, Artificial Sequence
  Description of Artificial Sequence Synthetic oligonucleotide
ggtacacggannnnnn

SEQ ID NO: 21 - DNA, 27 bp, Artificial Sequence
  Description of Artificial Sequence Synthetic oligonucleotide
nnnnnntccgtgtaccaaaaaaaaaaa

SEQ ID NO: 22 - DNA, 30 bp, Artificial Sequence; identical to SEQ ID NO: 14
  Description of Combined DNA/RNA Molecule Synthetic oligonucleotide

SEQ ID NO: 23 - DNA, 40 bp, Artificial Sequence
  Description of Artificial Sequence Synthetic oligonucleotide
nnnnnntccgtgtaccaagggtgccttttaaaaaaaaaaa